(19) United States
(12) Patent Application Publication
(10) Pub. No.: US 2013/0218073 A1
(43) Pub. Date: Aug. 22, 2013

(54) BLOOD ACCESS DEVICE

(71) Applicants: Olof Ekdahl, Lund (SE); Marco Zanini, Mirandola (SE); Per Hansson, Akarp (SE); Eddie Nilsson, Hoor (SE); Olof Jansson, Vellinge (SE); Lennart Jonsson, Bjarred (SE)

(72) Inventors: Olof Ekdahl, Lund (SE); Marco Zanini, Mirandola (SE); Per Hansson, Akarp (SE); Eddie Nilsson, Hoor (SE); Olof Jansson, Vellinge (SE); Lennart Jonsson, Bjarred (SE)

(21) Appl. No.: 13/806,039

(22) PCT Filed: Jun. 20, 2011
(86) PCT No.: PCT/EP2011/060230

§ 371 Date: May 8, 2013

Related U.S. Application Data
(60) Provisional application No. 61/358,025, filed on Jun. 24, 2010.

(30) Foreign Application Priority Data
Jun. 24, 2010 (SE) 1050697-0

(51) Int. Cl. A61M 5/168; A61M 1/36; A61M 25/02

(52) U.S. Cl. CPC A61M 5/168; A61M 25/02; A61M 1/3621
USPC 604/67; 604/245; 604/180

(57) ABSTRACT

A blood access device including a fluid conduit configured to convey a flow of fluid to a target vessel, a second lever element pivotally and resiliently connected to a first lever element such that it may move from a first position to a second position. A biasing force is exerted on the second lever element for moving it from the first position to the second position, and a blocking member may be activated by the second lever element, such that a flow of fluid is decreased when the second lever element is moved from the first position to the second position. The lever elements are configured to be fixed to the target vessel when the second lever element is in the first position, and the second lever element moves to the second position and activates the blocking member if the fixation is at least partially lost.

### TECHNICAL FIELD

The invention relates to a blood access device fitted with lever elements that may activate a blocker which thereby decreases a flow of fluid conveyed by the blood access device.

### BACKGROUND ART

In extracorporeal blood treatment blood is withdrawn from a patient, treated and then returned to the patient by means of an extracorporeal blood flow circuit. The blood is generally circulated through the circuit by one or several pumps, and the circuit is connected to a blood vessel access of the patient, typically via one or more access devices, such as needles or cannulas inserted into the blood vessel access. Depending on method of blood treatment, the blood may be withdrawn and returned via the same blood vessel access or via separate blood vessel accesses. Extracorporeal blood treatment include hemodialysis, hemodiafiltration, hemofiltration, plasmapheresis etc.

In extracorporeal blood treatment, it is important to reduce the risk of malfunction in the extracorporeal blood flow circuit, since this may lead to a potentially life threatening condition for the patient. For example, serious conditions may arise if the extracorporeal blood flow circuit is disrupted, e.g. if an access devices for blood reintroduction is dislodged from the blood vessel access, which may cause life threatening blood loss of the patient.

To address this problem available apparatuses for extracorporeal blood treatment often include surveillance (monitoring) mechanisms that monitor the integrity of the blood flow circuit and generates an alarm and/or take any other suitable action whenever a potentially dangerous situation is detected. Such surveillance mechanisms typically operate on measurement signals from one or more pressure sensors in the circuit. Conventionally, the surveillance is performed by comparing a measured pressure level with a threshold value and/or by monitoring the presence of air bubbles in the circuit. For example, failure in the blood extraction may cause air to be introduced into the circuit, which typically causes a decrease of the measured pressure, or a flow in the circuit may be restricted, which typically causes an increase of the measured pressure. Also, a failure in returning the blood to the patient may be detected as a decrease in the measured pressure. However, it may be difficult to set appropriate threshold values for determining if an increase or decrease in pressure levels corresponds to the above mentioned serious situations that should be detected by the surveillance mechanisms.

To solve the problems above a number of solutions have been proposed. For example, GB2448374A discloses a clamping mechanism for dialysis tubing, where the mechanism comprises a clamp and a pin which each has a respective hole for the tubing. If a needle attached to the tubing disconnects from a patient, the pin will slide into the clamp, and once the pin travels more than a certain distance a narrow portion of the pin will engage a recess. This causes jaws to clamp the tubing and thus stop a flow of blood there through. The jaws may be opened and the pin reset by pressing two arms.

The known clamping mechanism appears to be capable of assisting in solving problems related to a blood access device dislodging from a blood vessel access. However, the mechanism appears rather complex and cumbersome to handle.

### SUMMARY

It is an object of the invention to at least partly overcome one or more of the above-identified limitations of the prior art. In particular, it is an object to provide a blood access device that provides an alternative way of reducing a flow of blood if the blood access device is dislodged or disconnected from a target vessel. In further embodiments it is also an object to provide at simple yet reliable and user-friendly blood access device.

Hence a blood access device is provided, which comprises: a fluid conduit configured to convey a flow of fluid to a target vessel; a first lever element; a second lever element pivotally and resiliently connected to the first lever element such that it is movable in a direction from a first position to a second position in relation to the first lever element, and such that a biasing force is exerted on the second lever element for moving it from the first position to the second position; and a blocking member configured to be activated by the second lever element, such that a flow of fluid conveyed by the fluid conduit is decreased when the second lever element is moved from the first position to the second position. The lever elements are configured to be fixed at a surface of the target vessel when the second lever element is in the first position, such that the fixation to the surface counteracts the biasing force exerted on the second lever element, thereby allowing, if the fixation is at least partially lost, the second lever element to move to the second position and activate the blocking member.

As indicated, the second lever element is movable in a direction from a first position to a second position in relation to the first lever element. Since the movement is relative, this is the same as the first lever element being movable in a direction from one position to another position in relation to the second lever element. Thus, in its most general form the movement between the first position and the second position only describes the lever elements mutual movement.

The decrease caused by the blocking member may typically be in the range of 60-100% in comparison when the blocking device is un-activated. For the embodiments described below, the decrease of the flow of fluid conveyed by the fluid conduit is typically at least 80%. As will be shown, both the first and the second lever element may activate the blocking member.

The surface is not a part of the blood access device but it forms an important, functional role in that it allows for the lever elements to be fixed in a certain position in relation to each other (i.e. allows the second lever element to be fixed in the first position). The fixation is functional in the sense that a fixing device must not necessarily connect the lever elements to the surface; it suffices that some part of the blood access device is connected to the surface since such a connection may fix other parts at the surface (e.g. the lever elements). Thus, fixing the lever elements at the surface of the target vessel may be understood as arranging the lever elements in a certain position in relation to the surface.

Typically, this may mean that one or both of the lever elements are pressed against the surface by virtue of the fixation. The second lever element is then in the first position and the fixation (i.e. the pressing against the surface) may counteract the resilient connection between the lever elements. When the fixation is lost, at least one of the lever elements is no longer pressed to the surface and the lever elements may move in relation to each other (i.e. the second lever element may move to the second position) by virtue of the resilient connection.

For some embodiments the resilient connection between the lever elements always exerts the biasing force on the second lever element for moving it to the second position. It may then be said that the second position of the second lever element is its “natural” or “neutral” position.

The first and/or the second lever element may include other functionality, for example by forming a part of the fluid conduit. Moreover, the lever elements may be embodied as one unit, for example in the form of an elongated member with a lever center, where the lever elements are formed by one part on a respective side of the lever center about which the elements may pivot.

The fixation of the lever elements fixes the blood access device to the surface of the target vessel. Accordingly, if the blood access device is dislodged from the target vessel the fixation to the surface is typically lost. This makes the blood access device advantageous in that it may automatically stop a flow of blood.

The blood access device may comprise a resilient element connected to the lever elements for exerting the biasing force on the second lever element. This includes a possibility that the resilient element is connected to any of the lever elements via further connection elements. More specifically, the resilient element may be comprised in the blocking member.

The blood access device may comprise a needle arranged to be inserted in the target vessel, wherein the fluid conduit is configured to convey a flow of fluid to the target vessel via the needle.

The fluid conduit may comprise a flexible line, and the blocking member may be configured to at least partly occlude the flexible line when the second lever element is in the second position.

The blocking member may be arranged on the first and second lever elements, and the lever elements may be configured to press the blocking member against the flexible line, such that the flexible line is at least partially occluded when the second lever element is in the second position.

The blocking member may comprise a protrusion on the first lever element. Also, the blocking member may comprise a protrusion on the second lever element.

The blocking member may be arranged to be introduced into a fluid conduit of the center portion, such that a flow of fluid conveyed by the center portion is decreased when the lever element is moved from the first position to the second position. The blocking member may also be arranged to be introduced into the fluid conduit by the second lever element exerting a force on it, when the second lever element is moved from the first position to the second position.

The fluid conduit may comprise a seating configured to receive the blocking member.

The blood access device may comprise a removable locking device that is configured to prevent a movement of the second lever element from the first position to the second position.

The lever elements may comprise an adhesive configured to attach to the surface.

The blood access device may comprise a center portion connected to the fluid conduit, and each of the first lever element and the second lever element may have the form of a respective wing pivotally connected to the center portion, such that the second lever element is foldable from the first position to the second position, in a direction towards the first lever element.

Any of the lever elements may comprise an opening configured to receive a portion of the resilient element, when the second lever element is in the second position. Also, any of the lever elements may comprise a cut-out configured to abut against an abutment surface of the center portion, when the second lever element is in the second position.

The first and second lever elements may comprise a respective groove configured to act as hinges for the lever elements, such that the first and second lever elements are foldable in a direction towards each other.

The first lever element may be configured to pivot about a first pivot-axis and the second lever element may be configured to pivot about a second pivot-axis, when the second lever element moves from the first position to the second position. The first pivot-axis may be parallel with and offset from the second pivot-axis.

The first lever element may have the form of an elongated center portion that is connected to the fluid conduit.

The first lever element may extend along a first geometrical axis and the second lever element may comprise a portion that extends along a second geometrical axis, where the geometrical axes are substantially parallel when the second lever element is in the first position. The geometrical axes may extend along a plane of the surface, when the second lever element is in the first position. When the second lever element is in the second position the geometrical axes may be offset from a parallel direction.

The blood access device may comprise a set of wings configured to fix the first lever element to the surface. Also, the wings may comprise an adhesive configured to attach to the surface.

The blood access device may be configured to be connected to and cooperate with a blood treatment apparatus. This means that the blood access device may be deliberately and intentionally configured to operate in combination with a blood treatment apparatus, such that any requirements typically specific for blood treatment apparatus are fulfilled.

According to another aspect of the invention, a blood treatment apparatus is provided which comprises the inventive blood access device described above. The blood access device of the blood treatment apparatus may include any of the variants and features of the above described blood access device.

The blood treatment apparatus may comprise: a blood line connected to the blood access device; a pressure sensor arranged in the blood line and configured to measure a pressure in the blood line; and at least one processor unit. The processor unit is configured to receive a pressure reading from the pressure sensor, and respond to the pressure reading if the pressure reading is associated with the blocking member of the blood access device being activated. Of course, the processor unit may still respond to pressure variations related to various other conditions and situations. Exactly how much the pressure is changed when the blocking member is activated may be empirically determined, as the pressure change depends on the specific types of materials and components used for the blood access device.

### BRIEF DESCRIPTION OF THE DRAWINGS

Embodiments of the invention will now be described, by way of example, with reference to the accompanying schematic drawings, in which

FIG. 1 is a perspective view of a first embodiment of a blood access device in a first position that does not reduce a flow of blood,

FIG. 2 is a perspective view of the blood access device of FIG. 1, but in a second position that reduces a flow of blood,

FIG. 3 is a perspective view of the blood access device of FIG. 1, when it is fixed at a target vessel,

FIG. 4 is a perspective view of a second embodiment of a blood access device, in a position that does not reduce a flow of blood,

FIG. 5 is a perspective view of a third embodiment of a blood access device, in a position that does not reduce a flow of blood,

FIG. 6 is a perspective view of a fourth embodiment of a blood access device, in a first position that does not reduce a flow of blood,

FIG. 7 is a cross-sectional side view of the blood access device of FIG. 6,

FIG. 8 is a perspective view of the blood access device of FIG. 6, but in a second position that reduces a flow of blood,

FIG. 9 is a cross-sectional side view of the blood access device of FIG. 8,

FIG. 10 is a schematic view of a blood treatment apparatus that comprises two blood access devices for employing double-needle operation, and

FIG. 11 is a schematic view of a blood treatment apparatus that comprises one blood access device for employing single-needle operation.

### DETAILED DESCRIPTION

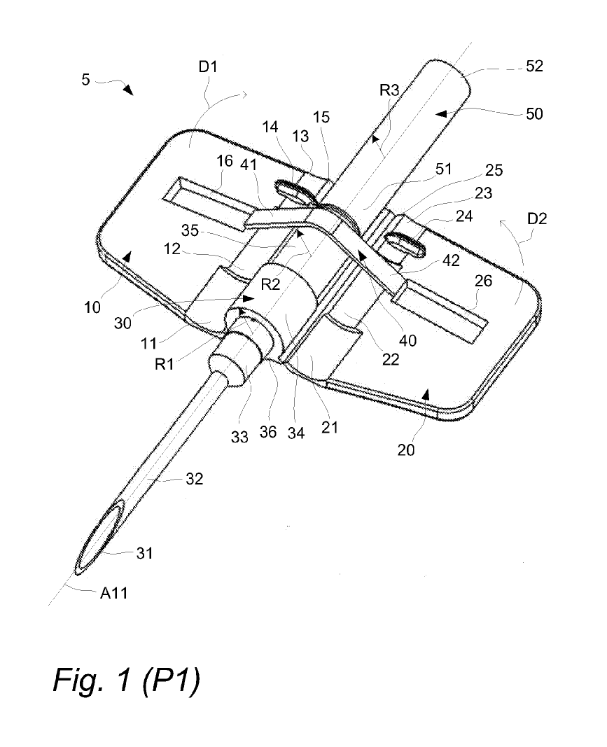

With reference to FIG. 1 a first embodiment of a blood access device 5 is illustrated which comprises a center portion 30 that extends along a first geometrical axis A11. A needle 32 with a sharp needle tip 31 is via a needle connector 33 attached to the center portion 30. At an end of the center portion 30 opposite the needle connector 33 is a fluid conduit 50 connected and is arranged to convey a flow of fluid to the center portion 30, which may convey the flow of fluid further to the needle 32 before it exits the blood access device 5 at the needle tip 31. Of course, a flow of fluid may also be reversed, i.e. fluid may be drawn at the needle tip needle tip 31, conveyed through the needle 32 and further through the center portion 30 and the fluid conduit 50. The fluid is typically treated and/or untreated blood, depending on how the blood access device 5 is applied.

In further detail, the center portion 30 has an elongated, cylindrical shape and may comprise a first section 34 closest to the needle connector 33 and defined by a first radial value R1, and second section 35 closest to the fluid conduit 50 and defined by a second radial value R2. The center portion 30 is hollow in a direction along the first geometrical axis A11, such that a fluid may flow inside and along the center portion 30. The center portion 30 is typically made of a suitable plastic material with elastomeric properties.

The fluid conduit 50 comprises in turn a first section 51 closest to the center portion 30 and an second section 52 opposite the first section 51, and may be defined by a third radial value R3. The fluid conduit 50 has a tube-like shape for allowing a flow of fluid to flow in the fluid conduit 50 to or from the center portion 30, and is typically a medical grade silicon tube that may be compressed such that a flow in the fluid conduit 50 is occluded i.e. fully or partially prevented. Accordingly, if the fluid conduit 50 is compressed, a flow of fluid may be stopped or reduced, depending on how well the fluid conduit 50 is compressed. However, if the compression is released, the fluid conduit 50 returns to its original shape by virtue of its flexibility. The fluid line may, for example, be part of a lineset that connects the blood access device 5 to a blood treatment apparatus.

To the center portion 30 a first lever element 10 and a second lever element 20 is attached. In detail, the first lever element 10 may be attached to the center portion 30 via a first groove 15 that acts as a hinge for allowing the first lever element 10 to move in relation to the center portion 30, the fluid conduit 50 and the second lever element 20. The direction of movement of the first lever element 10 is illustrated by the arrow D1. The second lever element 20 may in a corresponding manner be attached to the center portion 30 via a second groove 25 that acts as a hinge for allowing the second lever element 20 to move in relation to the center portion 30, the fluid conduit 50 and the first lever element 10. The direction of movement of the second lever element 20 is illustrated by the arrow D2. The first lever element 10 and the second lever element 20 may be attached to the center portion 30 at various positions along the center portion 30, such as at a lower section 36.

Thus, from this follows that the second lever element 20 is pivotally connected to the first lever element 10, which may be done either directly or via the center portion 30, such that it is movable in a direction D2 from a first position to a second position in relation to the first lever element 10. In this context it should be noted that movement of the first lever element 10, movement of the second lever element 20 or movement of both the first lever element 10 and the second lever element 20 always results in the second lever element 20 being moved in relation to the first lever element 10.

A resilient element 40 is at a first end 41 connected to the first lever element 10, extends over the center portion 30, and is at a second end 42 connected to the second lever element 20. The resilient element 40 may be made of a plastic material with elastomeric properties or may be embodied as a steel spring. The resilient element 40 biases the first lever element 10 and the second lever element 20 in a direction towards each other, for moving the lever elements 10, 20 in the directions D1 and D2, such that they may be folded over the center portion 30 and over the first section 51 of the fluid conduit 50, i.e. in a direction towards each other.

If the first lever element 10 and the second lever element 20 are fully folded towards each other, a first cut-out 11 of the first lever element 10 abuts against the first section 34 of the center portion 30. Also, a second cut-out 12 abuts against the second section 35 while a third cut-out 13 abuts against the first section 51 of the fluid conduit 50. In a corresponding manner, when fully folded against the first lever element 10, the second lever element 20 has corresponding cut-outs 21, 22 and 23 that abut against the sections 34, 35 of the center portion 30 and against the first section 51 of the fluid conduit 50. The cut-outs 11, 21 may be defined by a radius that corresponds to the first radial value R1, the cut-outs 12, 22 may be defined by a radius that corresponds to the second radial value R2, while the cut-outs 13, 23 may be defined by a radius that corresponds to the third radial value R3. In this context, the cut-outs 11, 12, 13, 21, 22 and 23 may be referred to as “abutment surfaces”.

Close to the fluid conduit 50 a blocking member in the form of a first protrusion 14 on the first lever element 10 and a second protrusion 24 on the second lever element 20 is arranged. If the lever elements 10, 20 are folded towards each other, the blocking member 14, 24 is pressed towards the first section 51 of the fluid conduit 50, which thereby compresses and hence occludes the fluid conduit 50 such that a flow of fluid there through is reduced.

Alternatively, the blocking member may comprise only one of the protrusions 14, 24, but occlusion of the fluid conduit might then be less efficient in comparison with using two protrusions.

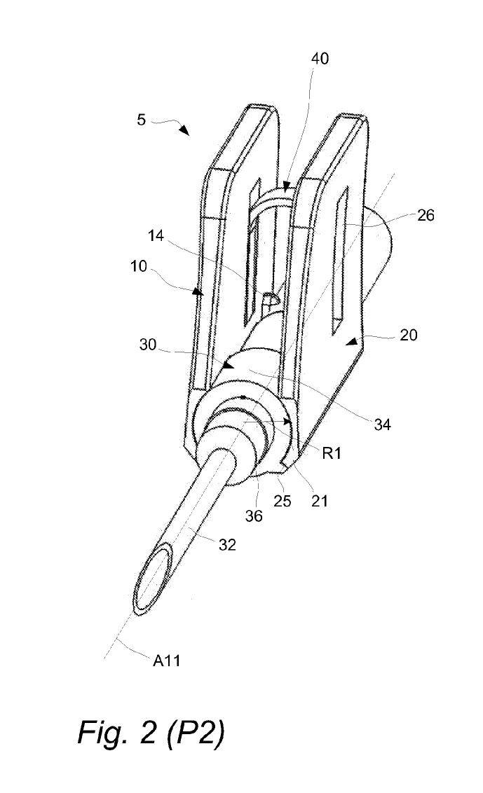

With further reference to FIG. 2, the blood access device 5 is illustrated in a second position P2 where the blocking member 14, 24 compresses the fluid conduit 50. This may be compared with FIG. 1, where the blood access device 5 is illustrated in a first position P1, and where the blocking member 14, 24 is far from the fluid conduit 50 which hence is fully open. Of course, when the blood access device 5 is in the first position P1 or second position P2, the second lever element 20 is also in the first position P1 respectively in the second position P2, as seen in relation to the first lever element 10. Accordingly, it may be said that the first and second positions P1, P2 indicate positions of any of the first lever element 10, the second lever element 20 and the blood access device 5 per se. However, for the most general definition of the positions P1, P2 used herein, the first position P1 primarily indicates a first position of the second lever element 20 in relation to the first lever element 10, and the second position P2 primarily indicates a second position of the second lever element 20 in relation to the first lever element 10.

Since the second lever element 20 is pivotally and resiliently (via the resilient element 40) connected to the first lever element 10, it is movable in the direction D2 from the first position P1 (FIG. 1) to the second position P2 (FIG. 2) in relation to the first lever element 10. Thus, the biasing force is exerted on the second lever element 20 for moving it from the first position P1 to the second position P2.

As evident from the Figures, additionally or alternatively, the first lever element 10 may just as well be seen as moving in relation to the second lever element 20, as it is only a matter of how a suitable form of coordinate system that describes the movements is defined.

From above follows that the blocking member in form of protrusion 24 is activated by the second lever element 20 such that a flow of fluid conveyed by the fluid conduit 50 is decreased when the second lever element 20 is moved from the first position P1 to the second position P2 (the protrusion 24 then compresses the fluid conduit). This does of course not exclude that also the blocking member in form of protrusion 14 is activated by the first lever element 10 such that it compresses the fluid conduit 50. In practice, the blocking member 14, 24 is activated by both lever elements 10, 20, which causes the protrusions to simultaneously compress the fluid conduit 50 from two opposite directions.

When the blood access device 5 is in the second position P2, the first end 41 of the resilient element 40 is partially received in an opening 16 in the first lever element 10 while the second end 42 of the resilient element 40 is partially received in an opening 26 in the second lever element 20. This allows the lever elements 10, 20 to fold closer against each other since the resilient element does not collide with any part of the levers 10, 20.

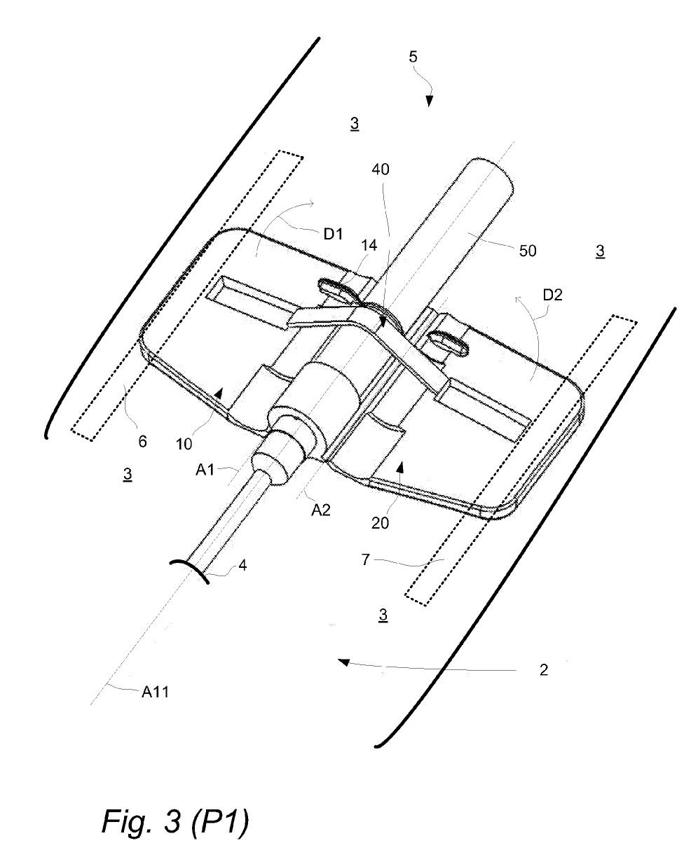

With further reference to FIG. 3, the blood access device 5 is illustrated when it is inserted into a target vessel 2. Examples of target vessels include containers of blood, human beings and animals. When the target vessel 2 represents a human being in form of a patient, the blood access device 5 is often inserted into a vein of the patient via a blood access 4. More specifically, the second lever element 20 is fixed at a surface 3 of the target vessel 2 when it is in the first position P1. In this embodiment, even though it is not necessary for all achievable embodiments, as will be described further on, the first lever element 10 may also be fixed at the surface 3. It may be said that the fixation causes the lever elements 10, 20 to be fixed in relation to the surface 2. An example of a common surface to which the blood access device may be fixated is an arm of a patient.

Suitable fixtures includes conventional bandages, plasters and adhesive tapes, which are schematically illustrated by a first fixture 6 that crosses the first lever element 10 and a second fixture 7 that crosses the second lever element 20. The fixture may also comprise an adhesive arranged on the lever elements 10, 20 on lever element surfaces facing the surface 3 or the target vessel 2. For this purpose any suitable and conventional adhesive may be applied on the lever elements (on the side intended to face e.g. a patient arms).

Even though the fixture as illustrated as crossing the lever elements 10, 20 and as parallel with the first geometrical axis A11, the fixture is advantageously arranged across both lever elements 10, 20 in a direction transverse the first geometrical axis A11. This is typically the case when the blood access device 5 is inserted into an arm of a patient, where the fixture may be wrapped several times around the arm and the blood access device 5.

The fixtures 6, 7 provide a fixation of the first lever element 10 and the second lever element 20 to the surface, which counteracts the biasing force exerted by the resilient element 40. If the fixation is lost the resilient element 40 moves the second lever element 20 from the first position P1 to the second position P2, which thereby activates the blocking member 14, 24 which in turn occludes the fluid conduit 50. In this context, regardless if only the fixation 6 or the fixation 7 comes off the surface 2, the second lever element 20 still moves in relation to the first element, i.e. from the first position P1 to the second position P2. If this happens, one of the lever elements 10, 20 may still be fixed to the surface 2.

Since the fixture holds the lever elements 10, 20 in place, it also holds the needle 32 in place. Thus, whenever the needle 32 is dislodged from the target vessel 2, the fixation to the surface 3 is also lost. Accordingly, whenever the needle 32 is dislodged the second lever element is automatically moved to the second position P2 which causes a flow of fluid to be reduced.

For illustrating the movement of the second lever element 20 from the first position P1 to the second position P2, the first element may then be arranged to pivot about a first pivot-axis A1, while the second lever element 20 may be arranged to pivot about a second pivot-axis A2. The pivot-axes A1, A2 may then be mutually parallel and offset. Also, for a more graphical representation of the lever elements 10, 20, it may be said that each of the lever elements has the form of a respective wing.

In other words, the blood access device 5 of FIGS. 1-3 may be said to comprise: a center portion in the form of the fluid conduit 50 for conveying a flow of fluid to a target vessel 2; a first wing 10 and a second wing 20 which are pivotally connected to the center portion 50 such that they are foldable from the first position P1 to the second position P2 in a direction D1, D2 towards each other, the wings configured to be in the first position when attached to the surface 3 of the target vessel 2; a blocking member 14, 24 configured to decrease, when the wings are in the second position P2, a flow of fluid conveyed by the fluid conduit 50; and a resilient element 40 connected to the wings 10, 20 and configured to bias the wings 10, 20 in the direction D1, D2 towards each other, such that the wings 10, 20 move to the second position P2 when any of the wings 10, 20 are detached from the surface 3 of the target vessel 2 and the blocking member 14, 24 thereby decreases a flow of fluid conveyed by the fluid conduit 50.

The resilient element 40 may have the form of an elastic strip made of plastics or may be e.g. a leaf spring. Alternatively or additionally, the resilient element may comprise a rubber string that connects the first lever element 10 with the second lever element 20. In any case, when inserting the blood access device in the target vessel, one must typically “open” the device in the sense that the lever elements are moved from each other, such that the second lever elements move from the second position to the first position. The lever elements may then be fixed to the surface and folds whenever a fixture is lost. The device must be “opened” since it is prior use generally stored in at least a partially folded position (approximately the second position).

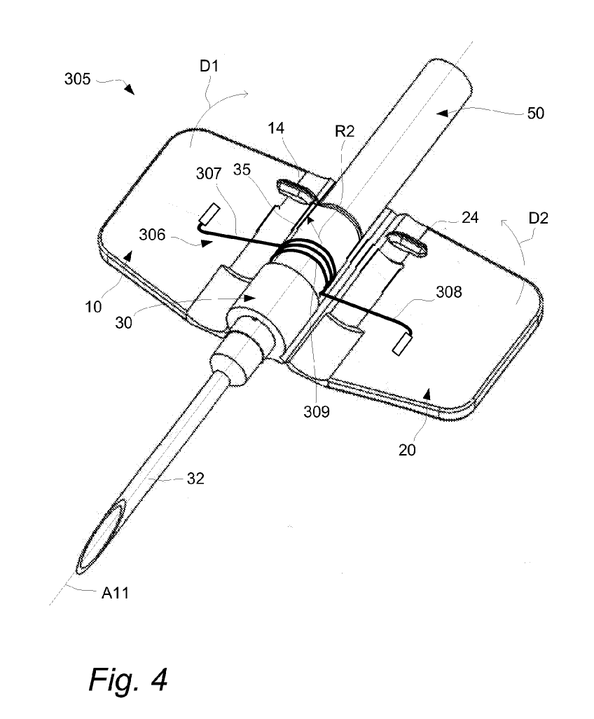

The first lever element 10 and the second lever element 20 may be resiliently connected to each other in other ways. For example and with reference to FIG. 4, in a second embodiment a blood access device 305 may have a resilient element in the form of a torsion spring 306 wound around the center portion 30 and having two arms 307, 308 that extend towards and connects to the first lever element 10 and the second lever element 20. As may be seen, the resilient element 306 basically represents the only difference between the second blood access device 305 and the previously described blood access device 5, as all other functionality remains the same. In principle, the resilient element 306 resembles a conventional spring used for clothespins, with a spring center 309 from which the arms 307, 308 extend.

Alternatively or additionally, the spring center 309 may form the blocking member. In this case the protrusions 14, 24 may be omitted or may be combined with the blocking member in form of the spring center 309. The blocking mechanism of the spring center 309 may then be based on a variable inner diameter of the spring center 309, where the variable inner diameter changes in dependence on the position of the lever elements 10, 20. Specifically, when the lever elements 10, 20 are in the first position P1 illustrated by FIG. 4, the arms 307, 308 expand the spring center 309 in the sense that its inner diameter is increased. When the lever elements 10, 20 are folded towards each other the arms 307, 308 are also folded towards each other, which causes the inner diameter of the spring center 309 to decrease and the second section 35 to be clamped by the spring center 309, such that a flow of fluid through the second section 35 is decreased. For achieving a proper clamping, the inner diameter of the spring center 309 may correspond to the outer diameter of the second section 35 when the lever elements 10, 20 are in the first position P1 (i.e. the diameter is then two times the second radial value R2).

In this alternative the second section 35 may have a tube-like form and is preferably made of a compressible material such as silicon. Exactly how much the diameter of the spring center 309 changes when the lever elements 10, 20 move depends on the specific configuration of the torsion spring 306. A suitable torsion spring configuration that results in a proper decrease of the flow of fluid may be empirically determined in combination with the selection of a suitable radius R2 for the second section 35. Modification of a spring (like the torsion spring 306) for achieving a certain increase/decrease of an inner diameter is per se known, and any suitable technique may be used. In operation, if a fixture is lost and the lever elements 10, 20 fold towards each other, the torsion spring 306 is not longer “counteracted” and may then clamp the second section 35.

From above follows that the torsion spring 306 may form both the resilient member as well as the blocking member.

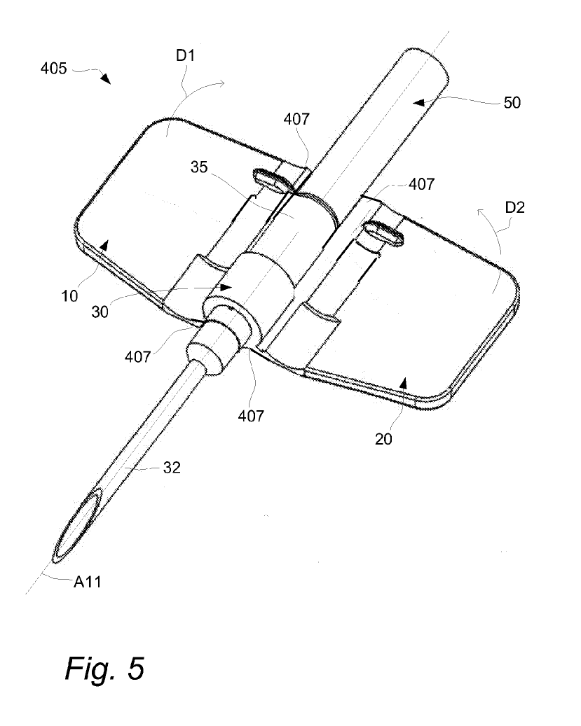

With further reference to FIG. 5, a third embodiment of a blood access device 405 may have an element in the form of a resilient or flexible section 407 that resiliently connects the first lever element 10 with the second lever element 20. In this embodiment the flexible section 407 may form a lower section of the blood access device 405, and may have a curvature (in an unloaded state) that corresponds to the lever elements 10, 20 being fully folded towards each other. Suitable materials for the section 407 are resilient plastics and steel springs.

Apart from the physical difference in resilient elements, the embodiments of FIGS. 1, 4 and 5 operate in a similar manner, since the resilient elements 40, 306 and 407 bias the lever elements in a corresponding manner and perform identical functionalities. Other types of resilient elements are of course conceivable. Also, the resilient element in the form of section 407 may be formed as an integral part of the first and/or second lever element, but still performs a resilient functionality and may for this reason be seen as a “resilient element”.

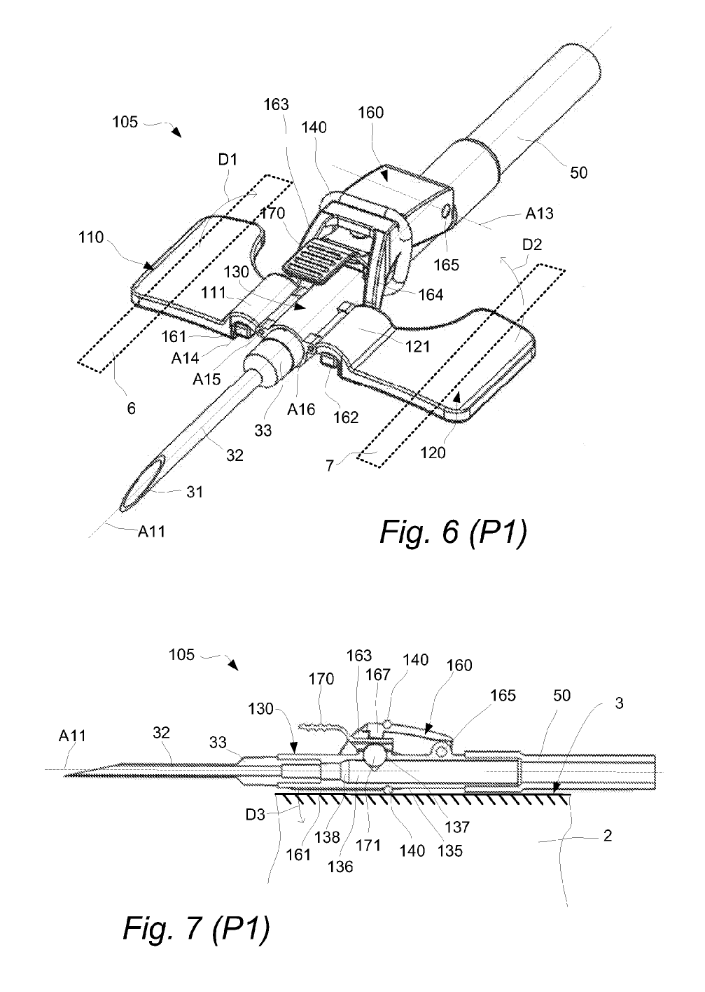

With further reference to FIGS. 6 and 7 a fourth embodiment of a blood access device 105 is illustrated, which comprises a center portion 130 that extends along a first geometrical axis A11. A needle connector 33 connects a needle 32 to the center portion 130, and at an opposite end of the center portion 130 a fluid conduit 50 is connected. Additionally, in this embodiment the center portion 130 has the form of a first lever element 130, and a second lever element 160 is pivotally connected to the first lever element 130 via a hinge 165. The second lever element 160 may thus pivot about a geometrical axis A13 that extends through the hinge 165. Hence, this embodiment differs from the embodiment of FIG. 1 in that the center portion acts as lever element by forming the “the first lever element 130”. The wing formed elements 10, 20 in FIGS. 1-5 should not be confused with the wings 110, 120 in FIGS. 6-9.

The first lever element 130, i.e. the center portion, has an elongated, pipe-like shape with a fluid line 136 that allows the first lever element 130 to convey fluid from a fluid conduit 50 to the needle 32 or vice versa. Here, a “fluid conduit” may be seen as the fluid line 136 in combination with the fluid conduit 50. The same applies for the previous embodiments, where the center portion 30 of e.g. FIG. 1 or the first lever element 130 of FIG. 6 in combination with the fluid conduit 50 may be seen as a common fluid conduit.

The second lever element 160 extends from the hinge 165 and above the first lever element 130 in a direction along the first geometrical axis A11. Two arms 163, 164 of the second lever element 160 extend further downwards towards the first lever element 130 and to a respective side of the first lever element 130, such that ends or portions 161, 162 of the arms 163, 164 are on a respective, opposite side of the first lever element 130.

A first wing 110 is connected to the first lever element 130 via a hinge that allows the first wing 110 to move about an axis A15, and a second wing 120 is connected to the first lever element 130 via another hinge that allows the second wing 120 to move about another axis A16. The wings 110, 120 stabilizes the blood access device 105 by fixing it more secure at a surface 3 of a target vessel 2, for example by fixing the wings 110, 120 to the surface 3 by means of fixtures 6, 7 that may be similar with the fixtures previously described. This includes the possibility of adding an adhesive to the wings for attaching them directly to the surface 3. Preferably each of the wings 110, 120 has a respective curvature 111, 121 so as not to engage with the arms 163, 164 of the second lever element 160.

It is possible to omit the wings 110, 120 while still fixing the lever elements 130 160 to the surface 3 of the target vessel 2. This may be accomplished by e.g. wrapping a fixture about the arm of a patient (the target vessel) and across the needle connector 33.

The second lever element 160 comprises a heel 167 that may engage a blocking member 171 in form of a ball that may be pushed into the fluid line 136 of the first lever element 130 (i.e. pushed into the center portion) via an opening 137. If a flow is present in a direction from the fluid conduit 50 to the needle 32, the blocking member 171 will travel along a flow direction and end at a seating 138 that receives the blocking member 171.

A locking device 170 is arranged between the second lever element 160 and the blocking member 171 for fixing a location of the second lever element 160 and thereby prevents the blocking member 171 from being engaged by the second lever element 160. When fixed by the locking device 170, the second lever element 160 is in a first position P1 in relation to the first lever element 130. This corresponds to a situation where the first lever element 130 extends along the first geometrical axis A11 and at least one of the portions 161, 162 extends along another geometrical axis A14 that is substantially parallel with the first geometrical axis A11.

Thus, when the second lever element 160 is in the first position P1, both the first lever element 130 and the second lever element 160 (via e.g. its portion 161) may extend at least partially along the surface 3 of the target vessel 2.

The locking device 170 is a safety measure. The locking device 170 must be removed (i.e. taken away from the blood access device 105) if the second lever element 160 shall be able to engage the blocking member 171. Thus, it shall be removed once the blood access device 105 is fixed to the surface 3.

If the fixture is lost, for example if the wings 110, 120 are detached from the surface 3, then the second lever element 160 is moved from the first position P1 to a second position P2, in a direction indicated by D3.

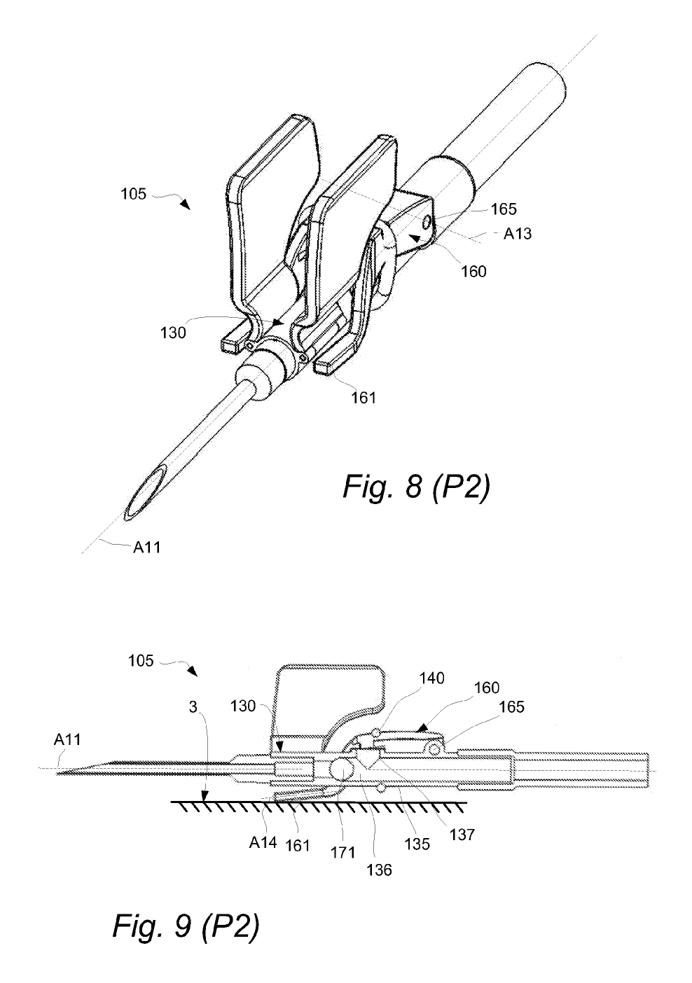

This situation is illustrated by FIGS. 8 and 9 and may as mentioned only occur if the locking device 170 has been removed. As may be seen, in the second position P2 the second lever element 160 has pivoted about the hinge 165, thereby pressing the blocking member 171 into the fluid line 136. The pivoting movement is caused by a resilient element 140 that connects the first lever element 130 to the second lever element 160, and thereby biases the second lever element 160 for moving it from the first position P1 to the second position P2, along the direction D3.

Accordingly, when the lever elements 130, 160 are fixed at the surface 3, e.g. by means of adhesive tape across the blood access device 105 or by means of the wings 110, 120, then the second lever element 160 is in the first position P1 by virtue of the surface 3 of the target vessel 2 applying a counteracting force on the second lever element 160. In other words, the fixation to the surface 3 counteracts the biasing force exerted on the second lever element 160 by the resilient element 140. If the fixation is lost, the biasing force is not counteracted and the second lever element 160 moves to the second position P2, which activates the blocking member 171. As with previous embodiments, the needle is not dislodged when the fixture is in place, while a dislodgement of the needle means that the fixture is lost which in turn means that the second lever element may move to the second position, and thereby cause a reduction of the flow of fluid in the fluid conduit.

Each embodiment of the blood access devices is advantageous in that a flow of blood is automatically stopped or at least reduced if the blood access device (and typically also the needle) loses its fixture to a target surface. Moreover, during tests it has been observed that the embodiments of the blood access device cooperate well with present blood treatment apparatuses, based on an actual reduction of a flow of blood through the device.

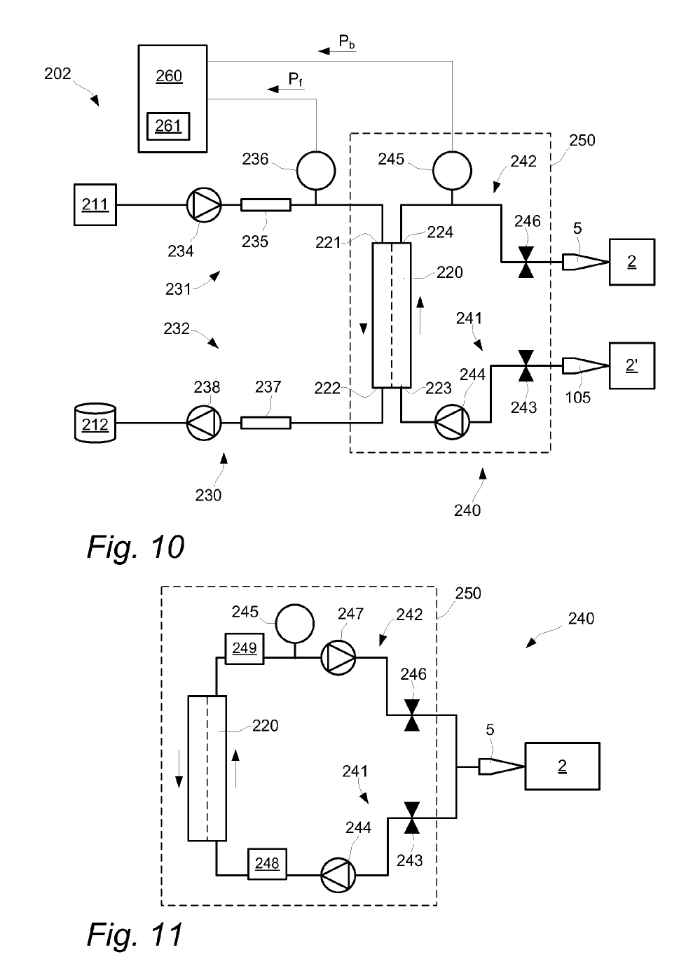

For illustrating the cooperation between a blood treatment apparatus 202 and any of the embodiments of the blood access device 5, reference is made to FIG. 10. It should here be noted that the blood treatment apparatus 202 cooperates equally efficient with all embodiments of blood access devices described herein.

In detail, the blood treatment apparatus 202 comprises a fluid line 230 that passes blood treatment fluid (sometimes referred to as “dialysis fluid”) from a fluid source 211 to a sink 212. The fluid line 230 has an upstream section 231 that comprises a pump 234 for conveying the fluid, a flow meter 235 and a pressure meter 236 that may read a pressure P_{f} in the fluid line. The upstream fluid line 231 is connected to a fluid inlet 221 of a blood treatment unit 220. The fluid line 230 has also a downstream section 232 that includes a flow meter 237 and a pump 238, and is upstream connected to an outlet 222 of the blood treatment unit 220 and is downstream connected to the sink 212.

Further, the blood treatment apparatus 202 comprises a blood line 240 which also is divided into an upstream section 241 and a downstream section 242. The upstream section 241 draws blood from a target vessel 2′ via a blood access device 105 of a type described above, comprises a clamp 243 and utilizes a blood pump 244 for feeding the blood to a blood inlet 223 of the blood treatment unit 220. The blood exits the blood treatment unit 220 via a blood outlet 224 to which the downstream section 242 is connected, and is passed back to a target vessel 2 via a further blood access device 5 of a type described above. The downstream section 242 comprises a clamp 246 and a pressure sensor 245 for measuring a pressure P_{b} in the blood line 240. The target vessel 2 may represent a vessel with treated blood and the target vessel 2′ may represent a vessel with untreated blood. Also, the target vessels 2, 2′ may in combination bee seen as one target vessel, for example if they represent a patient.

Apart from the blood access devices 5, 105 per se and the cooperation with the blood access devices, the blood treatment apparatus 202 includes conventional components and operates in a manner known within the art. In fact, when adapted according to the description below, basically any blood treatment apparatus with pressure sensors in the blood line may be used in connection with the blood access device described herein, while still obtaining additional advantages related to monitoring of dislodged blood access devices.

In detail, the blood treatment apparatus 202 comprises at least one processor unit 260 configured to i) receive (or acquire) a pressure reading P_{b} from the pressure sensor 245 in the blood line 240, and ii) respond to the pressure reading P_{b} if it corresponds to a blocking member of the blood access device being activated.

A suitable threshold value that may trigger the response may be empirically determined and depends generally on the specific type of blood access device that is employed. In any case, an activated blocking member generates a substantial pressure change that in experiments have shown relatively easy to identify. The response may, for example, include stopping the blood pump 244 and shutting of the clamps 243, 246, which efficiently reduces a loss of blood in case the blood access device comes loose from a target vessel.

Instead of, or as an alternative to, acquiring a pressure reading P_{b} from the pressure sensor 245 in the blood line 240, a pressure reading P_{f} may be acquired from the pressure sensor 235 in the fluid line 230. The processor unit 260 may then respond to the pressure reading P_{f} in the fluid line 230 if it has a value that corresponds to, or is associated with, activation of a blocking member of the blood access device. In principle, acquiring the pressure reading P_{f} in the fluid line 230 and responding thereto may be done in a manner similar with the operations in connection with the pressure reading P_{b} in the fluid line. Using the pressure reading P_{f} in the fluid line is an alternative or an addition to the use of the pressure reading P_{b} in the blood line and is relevant since a pressure change in the blood line 240 is typically transferred over the membrane of the blood treatment unit 220 and to the fluid line 230. Suitable threshold values for a pressure reading P_{f} in the fluid line that corresponds to the blood access device dislodging may be empirically determined.

For implementing the monitoring performed by the processor unit 260 it may include software instructions in form of e.g. computer program code for carrying out the monitoring, which may be stored in a computer readable medium 261 connected to the processing unit 260. Embodiments of the monitoring may for development convenience be written in a high-level programming language such as Java, C, and/or C++ but also in other programming languages, such as, but not limited to, interpreted languages. The software instructions may also be written in assembly language or even micro-code to enhance performance and/or memory usage. It will be further appreciated that the functionality of any or all of the monitoring performed by the blood treatment apparatus 202 may also be implemented using discrete hardware components, one or more application specific integrated circuits, or a programmed digital signal processor or microcontroller. Accordingly, the computer-readable medium 261 may store processing (software) instructions that, when executed by the processor unit 260, performs the above described monitoring of dislodgement of a blood access device.

The blood treatment apparatus 202 FIG. 10 illustrates double-needle operation. However, with further reference to FIG. 11, the embodiments of the blood access device may also be used with a blood treatment apparatus that operates in a single-needle mode. The blood treatment apparatus then comprises the same components as described in connection with FIG. 10, with the difference that a common blood access device 5 is used for cyclically drawing blood and returning blood to the target vessel 2. Even though one blood pump suffices, an extra blood pump 247 is arranged in the downstream section 242 for providing assistance in conveying the blood. At least one buffer volume should be included for obtaining a proper blood flow, such as a first buffer chamber 248 in the upstream section 241 and/or a second buffer chamber 249 in the downstream section 242. The procedure of single-needle operation may be performed in a conventional manner, and the acquiring of the pressure reading P_{b} or P_{f} and the response thereto may be performed in a manner similar to the procedure for the double-needle operation.

As the skilled person realizes, the various embodiments of blood access devices described herein may be combined in numeral ways. For example, various kinds of or principles for the resilient relationship between the first lever element and the second lever element may be employed. Also, the different principles of the blocking members may be adopted for each of the embodiments, for example by letting the fourth embodiment occlude the fluid conduit by clamping, while the first embodiment may use introduction of a blocking member into the conduit, instead of clamping the conduit. Thus, although various embodiments of the invention have been described and shown, the invention is not restricted thereto, but may also be embodied in other ways within the scope of the subject-matter defined by the claims.

1. A blood access device comprising:
a fluid conduit configured to convey a flow of fluid to a target vessel,
a first lever element,
a second lever element pivotally and resiliently connected to the first lever element such that the second lever element is movable in a direction from a first position to a second position in relation to the first lever element, and a biasing force is exerted on the second lever element for moving the lever element from the first position to the second position,
a blocking member configured to be activated by the second lever element, such that a flow of fluid conveyed by the fluid conduit is decreased when the second lever element is moved from the first position to the second position, wherein
the first and second lever elements are configured to be fixed at a surface of the target vessel when the second lever element is in the first position, such that the fixation to the surface counteracts the biasing force exerted on the second lever element, thereby allowing the second lever element to move to the second position and activate the blocking member if the fixation is at least partially lost.

2. A blood access device according to claim 1, comprising a resilient element connected to the first and second lever elements for exerting the biasing force on the second lever element.

3. A blood access device according to claim 1, comprising a needle arranged to be inserted in the target vessel, the fluid conduit configured to convey a flow of fluid to the target vessel via the needle.

4. A blood access device according to claim 1, wherein the fluid conduit comprises a flexible line, the blocking member configured to at least partly occlude the flexible line when the second lever element is in the second position.

5. A blood access device according to claim 4, wherein the blocking member is arranged on the first and second lever elements, the first and second lever elements are configured to press the blocking member against the flexible line, such that the line is at least partially occluded when the second lever element is in the second position.

6. A blood access device according to claim 1, wherein the blocking member comprises a protrusion on the first lever element.

7. A blood access device according to claim 1, wherein the blocking member comprises a protrusion on the second lever element.

8. A blood access device according to claim 1, wherein the blocking member is arranged to be introduced into a fluid line of the center portion, such that a flow of fluid conveyed by the center portion is decreased when the lever element is moved from the first position to the second position.

9. A blood access device according to claim 8, wherein the blocking member is arranged to be introduced into the fluid line by the second lever element exerting a force on the blocking member, when the second lever element is moved from the first position to the second position.

10. A blood access device according to claim 1, wherein the fluid conduit comprises a seating configured to receive the blocking member.

11. A blood access device according to claim 1, comprising a removable locking device configured to prevent a movement of the second lever element from the first position to the second position.

12. A blood access device according to claim 1, wherein the lever elements comprises an adhesive configured to attach to the surface.

13. A blood access device according to claim 1, comprising a center portion connected to the fluid conduit, each of the first lever element and the second lever element having the form a respective wing pivotally connected to the center portion, such that second lever element is foldable from the first position to the second position, in a direction towards the first lever element.

14. A blood access device according to claim 2, wherein any of the first and second lever elements comprises an opening configured to receive a portion of the resilient element, when the second lever element is in the second position.

15. A blood access device according to claim 13, wherein any of the first and second lever elements comprises a cut-out configured to abut against an abutment surface of the center portion, when the second lever element is in the second position.

16. A blood access device according to claim 13, wherein the first and second lever elements comprise a respective groove, such that the first and second lever elements are foldable in a direction towards each other.

17. A blood access device according to claim 13, wherein the first lever element is configured to pivot about a first pivot-axis and the second lever element is configured to pivot about a second pivot-axis, when the second lever element moves from the first position to the second position.

18. A blood access device according to claim 17, wherein the first pivot-axis is parallel with and offset from the second pivot-axis.

19. A blood access device according to claim 1, wherein the first lever element has the form of an elongated center portion connected to the fluid conduit.

20. A blood access device according to claim 18, wherein the first lever element extends along a first geometrical axis and the second lever element comprises a portion that extends along a second geometrical axis, the geometrical axes being substantially parallel when the second lever element is in the first position.

21. A blood access device according to claim 20, wherein the geometrical axes extend along a plane of the surface, when the second lever element is in the first position.

22. A blood access device according to claim 20, wherein the geometrical axes are offset from a parallel direction, when the second lever element is in the second position.

23. A blood access device according to claim 19, comprising a set of wings configured to fix the first lever element to the surface.

24. A blood access device according to claim 19, wherein the wings comprises an adhesive configured to attach to the surface.

25. A blood access device according to claim 1, configured to be connected to and cooperate with a blood treatment apparatus.

26. A blood access device according to claim 2 wherein the resilient member comprises a spring center that forms the blocking member.

27. A blood access device according to claim 26 wherein a blocking mechanism of the spring center is provided by a variable inner diameter of the spring center, where the variable inner diameter is changed in dependence on the position of the lever elements.

28. (canceled)

29. A blood treatment apparatus comprising:
the blood access device recited in claim 1,
wherein the blood treatment apparatus further comprises a blood connected to the blood access device,
a pressure sensor arranged in the blood line and configured to measure a pressure in the blood line, and
at least one processor unit is configured to receive a pressure reading from the pressure sensor, and respond to the pressure reading if it is associated with the blocking member of the blood access device being activated.

30. A blood access device comprising:
a fluid conduit having an internal flow passage configured to convey a fluid to a target vessel;
a first lever element connected to the fluid conduit;
a second lever element pivotally connected to at least one of the first lever element and the fluid conduit, wherein the second lever element pivots with respect to the first lever element and the fluid conduit, wherein the first and the second lever elements are configured to be fixed to a surface proximate the target vessel while the second lever element is in the first pivot position;
a blocking member adjacent the fluid conduit and moved by the second lever element, wherein the blocking member decreases the internal flow passage of the fluid conduit as the second lever elements pivots from the first pivot position to the second pivot position, and
a biasing component applying a biasing force to move the second lever element from the first pivot position to the second pivot position, and the biasing force is insufficient to move the second lever element to the second pivot position while the first and second lever elements are fixed to the surface and is sufficient to move the second lever element to the second pivot position when at least one of the first and second lever elements are released from being fixated to the surface.

31. The blood access device according to claim 30 further comprising a needle arranged to be inserted in the target vessel, and the fluid conduit configured to convey a flow of fluid to the target vessel via the needle.

32. The blood access device according to claim 30 wherein the fluid conduit comprises a flexible line and the blocking member is configured to at least partly occlude the flexible line when the second lever element is in the second pivot position.

33. The blood access device according to claim 32 wherein the blocking member is arranged on the first and second lever elements, the first and second lever elements are configured to clamp the blocking member against the flexible line.

34. The blood access device according to claim 30 further comprising a center portion connected to the fluid conduit, each of the first lever element and the second lever element having a respective wing pivotally connected to the center portion, such that the second lever element is foldable from the first pivot position to the second pivot position.

35. The blood access device according to claim 30, wherein the first lever element extends along a first geometrical axis and the second lever element comprises a portion that extends along a second geometrical axis, the geometrical axes being substantially parallel when the second lever element is in the first position.
